(19) United States
(12) Patent Application Publication
(10) Pub. No.: US 2017/0258486 A1
(43) Pub. Date: Sep. 14, 2017

(54) PERIOSTEUM ELEVATION TIP AND METHOD OF USE

(71) Applicant: Piezosurgery Inc., Columbus, OH (US)

(72) Inventors: Tomaso Vercellotti, Genova (IT); Constantin von See, Rosengarten (DE)

(21) Appl. No.: 15/067,014

(22) Filed: Mar. 10, 2016

(51) Int. Cl. A61B 17/32

(52) U.S. Cl. CPC A61B 17/320068; A61B 2017/320084; A61B 2017/320044

(57) ABSTRACT

An insert for use with a vibrating surgical tool has a proximal end for mounting to the vibrating surgical tool and a shaft extending from the proximal end. A head is mounted to the shaft, is curved along the longitudinal axis, and the terminal end thereof defines an edge for disposing against an incision between bone and periosteum. A passage is formed through the shaft for water to be forced through the passage and out of apertures formed on opposite sides of the insert. The edge is formed on the terminal end of the head, which is formed on a curve from one lateral side to an opposite lateral side. The edge extends from a front face to a tapered rear surface. A front face of the head forms an angle with a longitudinal axis of the shaft.

### CROSS-REFERENCES TO RELATED APPLICATIONS

(Not Applicable)

### STATEMENT REGARDING FEDERALLY-SPONSORED RESEARCH AND DEVELOPMENT

(Not Applicable)

### THE NAMES OF THE PARTIES TO A JOINT RESEARCH AGREEMENT

(Not Applicable)

### REFERENCE TO AN APPENDIX

(Not Applicable)

### BACKGROUND OF THE INVENTION

This invention relates generally to the field of piezoelectric surgical devices and surgical tips thereof, and more specifically to a periosteal preparation tip and method of use thereof in a piezoelectric surgical device.

The periosteum is a membrane that covers the outer surface of bones in humans and animal, except at the joints of long bones. The periosteum consists of dense irregular, connective tissue that is divided into layers, and is attached to bone by collagen fibers in the bone matrix. The periosteum provides an attachment for muscles and tendons to the bone, as well as nourishment by providing the blood supply to the body from the marrow.

Periosteal damage can be caused by the separation of the periosteum from the bone during surgery. Surgical procedures often have adverse effects on the periosteum due to the manner in which the periosteum is prepared by separation from the bone. The preparation of the periosteum is a routine procedure, and is commonly performed by using a surgical elevator. In such a procedure, the surgeon uses a scalpel to cut through soft tissue to the bone, and then uses the surgical elevator to peel the periosteum from the bone. Periosteal elevators, which are specialized instruments used for separating the periosteum from the bone, are also commonly used to lift full thickness soft tissue flaps. Periosteal elevators allow a surgeon to manually lift and separate periosteal tissue from the bone by inserting the blade of the elevator between the periosteum and the bone.

It is difficult or impossible for surgeons to prepare the periosteum in such a way that it remains unharmed using current tools and methods, and therefore this procedure often causes damage to the cells of the osteogenic layer. Conventional preparation instruments do not detach the periosteum as a whole from the bone, but instead disrupt its integrity by tearing it. Such instruments, and the procedures in which they are used, damage important regenerative cells of the periosteum, which are thus no longer available to provide nourishment to the bone they cover.

Ultrasonic instruments, which use high frequency vibrations, have been available for decades. Devices utilizing the piezoelectric effect for surgical purposes have been used since about 2000. Applications of piezoelectric devices include hard-tissue surgery, periodontal surgery, the removal of impacted teeth, apical surgery, and bone expansion. U.S. Pat. No. 6,695,847 to Bianchetti et al. describes a piezoelectric surgical device, and is hereby incorporated by reference.

Piezoelectric surgery tools have become an established technique for dental and other surgeries. The characteristic feature of the piezoelectric surgical tool system is preservation of soft tissue, because mineralized tissues are altered by frequencies above 20 kHz while soft tissue (e.g. nerves) damage occurs only by vibrations at frequencies above 50 kHz.

Piezoelectric devices can cut a specific type of tissue without causing damage to adjacent tissues, based on the frequency of vibration. In addition, piezoelectric devices have the advantage of causing minimal bleeding when they are used to cut bone. This reduces the risk of postoperative infections.

### BRIEF SUMMARY OF THE INVENTION

Piezoelectric surgical devices vibrate an instrument at high frequency to affect human and animal tissue. Such devices utilize a variety of removable tips to perform a variety of different functions, and the tips may be operated at various frequencies (or ranges of frequency) in order to have different effects on different types of tissue, including without limitation, bone, teeth, muscle, connective tissue and others. This is explained in more detail in U.S. Pat. No. 6,695,847 to Bianchetti et al., which is incorporated by reference herein.

A tip (which can also be called an “insert”, among other names) is removably mounted to the main body of the piezoelectric apparatus that is comfortably held by a human hand, and the ultrasonic vibrations generated thereby are transmitted to the tip. When the tip makes contact with the tissue that is to be affected, the rapid movement of the tip in contact with the tissue has the desired effect on the tissue. When the instrument is used properly, the surrounding tissue that is not to be affected by the chosen tip and frequency range is not harmed, even if the surrounding tissue is contacted by the tip.

Disclosed herein is an insert and a method of using the insert. The insert is designed for use with a vibrating surgical tool, and has a proximal end for mounting to the vibrating surgical tool and a shaft extending from the proximal end. A head is mounted to the shaft and has a head thickness substantially less than the head width. The head is curved through the head thickness, along the longitudinal axis of the insert, and the terminal end of the head defines an edge for disposing against an incision between bone and covering periosteum.

In a preferred embodiment, the tip includes a passage formed through the shaft to permit water or another liquid to be forced through the passage. At least one liquid-dispensing aperture formed on one side of the insert is in fluid communication with the liquid passage. In another embodiment, a second liquid-dispensing aperture is in fluid communication with the liquid passage and is formed on a second side of the insert opposite the first side for dispensing liquid from the second liquid-dispensing aperture. The liquid dispensing apertures may be formed on the head adjacent the shaft, and may be formed on opposite sides of the head.

The edge may be formed on the terminal end of the head, and may be along a curve extending from one lateral side to an opposite lateral side so that the terminal end of the insert is the apex of the curve and is centrally located on the insert. The edge may extend from a front face to a tapered rear surface and may be at least about 1.0 millimeter wide. A front face of the head may form an angle with a longitudinal axis of the shaft of about 28 degrees.

Disclosed herein is a method of separating at least a portion of a periosteum from a bone. The method comprises the step of mounting a removable insert on a vibrating surgical tool. The insert has a proximal end removably mounted to the vibrating surgical tool and a shaft extending from the proximal end. A head mounts to the shaft and the head has a head thickness substantially less than a head width. The head curves through the head thickness along the longitudinal axis of the insert, and a terminal end of the head defines an edge. Another step of the method is vibrating the tip at high frequency. The edge is disposed against an incision between bone and a periosteum covering the bone and manually displaced along the length of the incision.

The method may include forcing a liquid through the shaft and out of the insert adjacent the head on opposing sides of the head. The method may include disposing the longitudinal axis at an angle of between about 0 and 90 degrees relative to the incision, and may include disposing the longitudinal axis of the shaft at an angle of between about 15 and 30 degrees relative to the incision.

### BRIEF DESCRIPTION OF THE SEVERAL VIEWS OF THE DRAWINGS

FIG. 1 is a view in perspective illustrating an embodiment of the present invention.

FIG. 2 is another view in perspective illustrating the embodiment of FIG. 1.

FIG. 3 is a front view illustrating the embodiment of FIG. 1.

FIG. 4 is a side view illustrating the embodiment of FIG. 1.

FIG. 5 is a magnified view in perspective illustrating the embodiment of FIG. 1.

FIG. 6 is a bottom view in perspective illustrating the embodiment of FIG. 1.

FIG. 7 is a top view in perspective illustrating the embodiment of FIG. 1.

FIG. 8 is a section view through the line A-A of FIG. 3.

FIG. 9 is a magnified section view of the head of the embodiment of FIG. 1 through the line B-B of FIG. 1.

FIG. 10 is a front view illustrating the head of the embodiment of FIG. 1.

FIG. 11 is a photograph of the embodiment of FIG. 1 in an operable position.

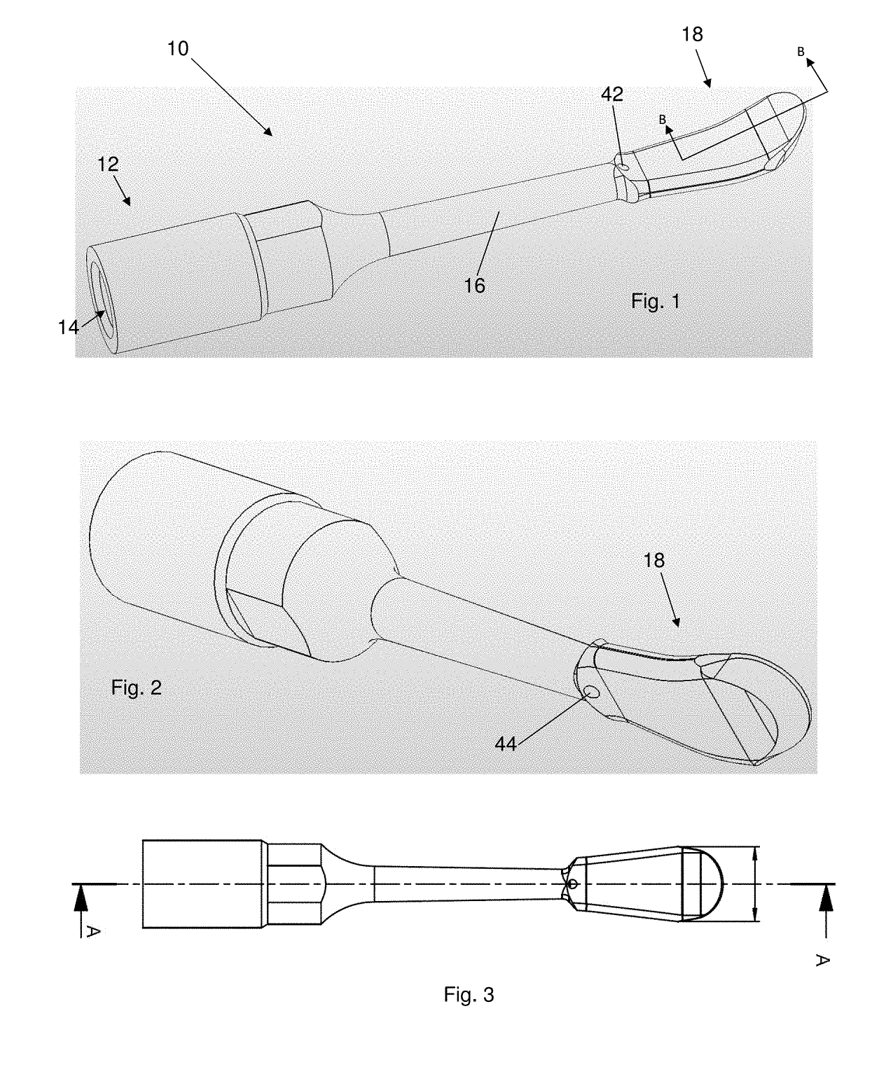

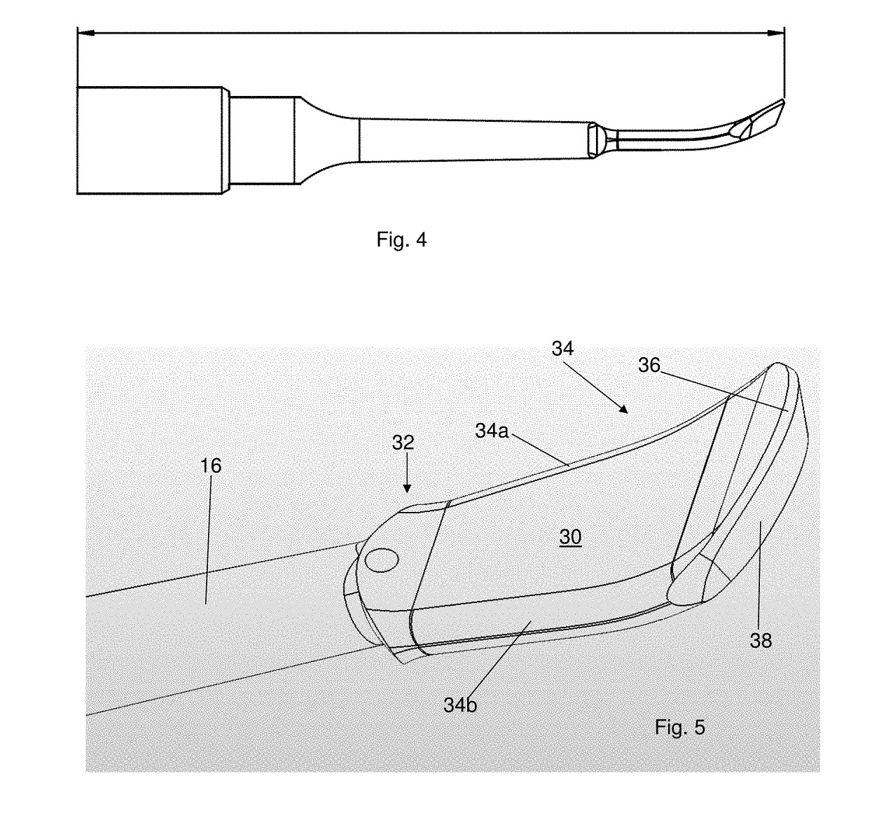

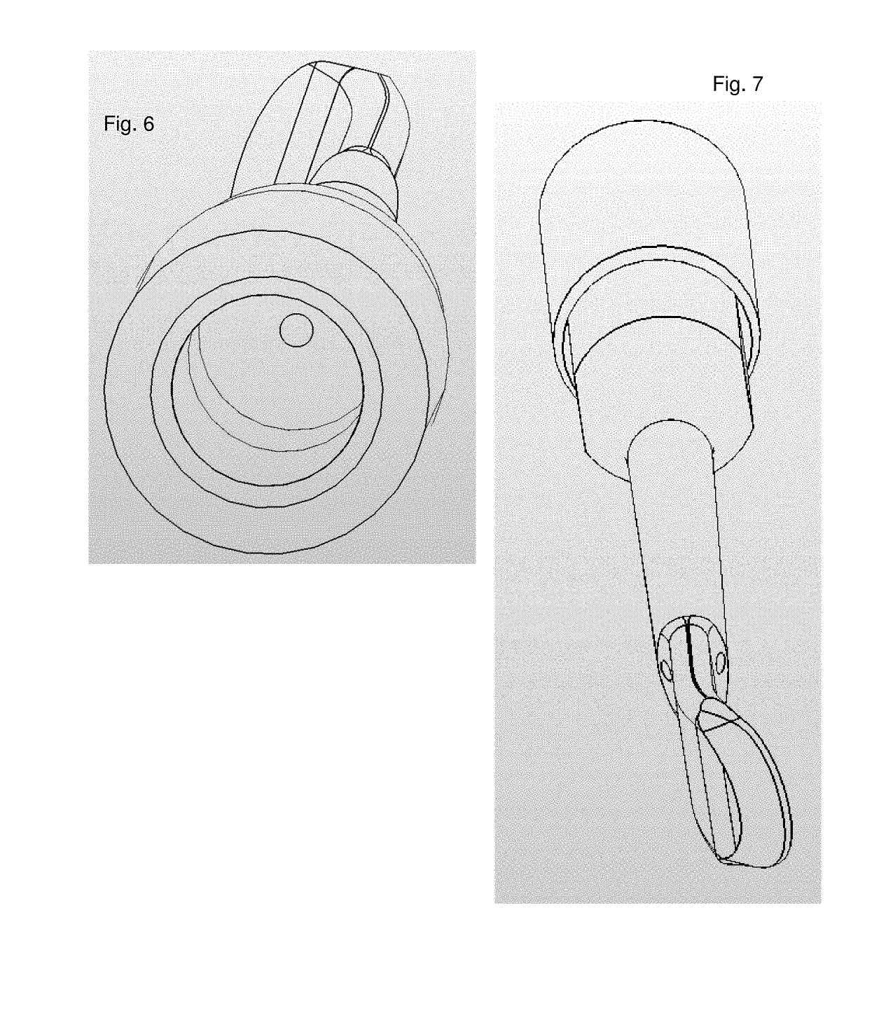

In describing the preferred embodiment of the invention, which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

The insert 10 shown in FIG. 1 may be mounted to a conventional vibrating surgical tool, such as the piezoelectric surgical device described in U.S. Pat. No. 6,695,847, in a manner in which conventional inserts described therein are mounted, such as by friction fit, threading, a collar that clamps the insert 10, or any other means. The insert 10 has a proximal end 12 with a bore 14 formed therein, a shaft 16, and a distal end 18 having a head 30. The proximal end 12 is mounted to the piezoelectric surgical device with the tissue-contacting head 30 disposed farther away from the piezoelectric surgical device than any other portion of the insert 10. Thus, the head 30 is at the end of the insert 10 that is conveniently placed by a surgeon in contact with human or other living tissue during its designed use. This may be during periodontal surgery, or any other surgery on humans or animals. It is also contemplated that the tissue may not be living, such as in the example of an autopsy in which minimal damage to the periosteum is desired.

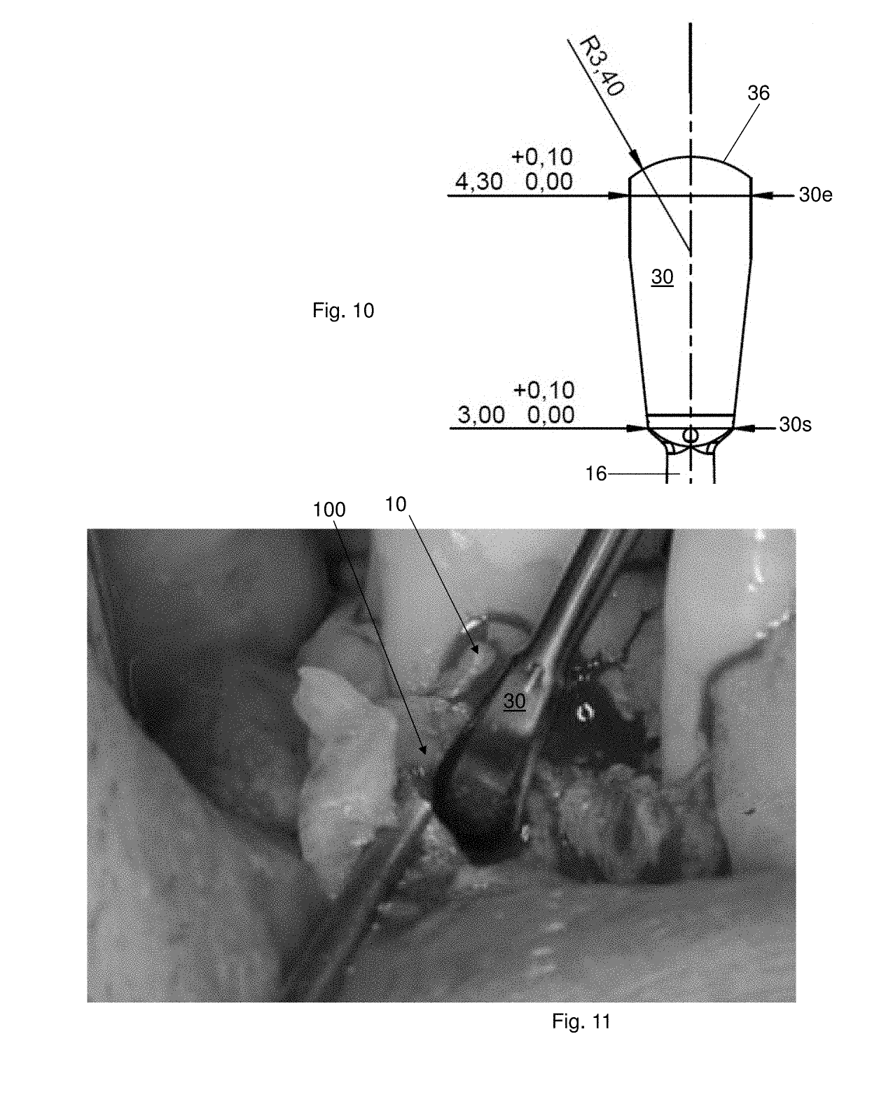

The head 30 is shown in a magnified view in FIG. 5 connected to the shaft 16 at a shaft end 32 of the head. The preferred width 30s (FIG. 10) of the head adjacent the shaft 16 is about 3.00 millimeters, and this can vary by at least one millimeter greater or lesser. An edge 36 is formed along a curved surface at an end of the head that is opposite from the shaft 16, and this edge 36 defines the terminal end of the head 30. As show in FIG. 10, the preferred width 30e of the head adjacent the edge 36 is about 4.30 millimeters, and this can vary by at least one millimeter smaller or larger. The edge 36 is formed on a preferably curved terminal portion of the head 30, which curves from one lateral side to the opposite lateral side. As shown in FIG. 10, the lateral radius of curvature of this edge 36 is about 3.4 millimeters, and may vary larger or smaller by 0.1 to 0.2 millimeters from the preferred radius for this embodiment. As shown in FIG. 9, the edge 36 is about 0.2 mm wide, and this thickness may vary by a fraction of a millimeter (+0.2 mm/−0.1 mm). The edge 36 meets the front face of the head 30 at an angle of about 90 degrees, and this angle may vary +/−5° from the preferred without substantially departing from the characteristics of the preferred embodiment. Thus, the head 30 has a thickness that is substantially less than the width of the head 30, and this configuration provides the curved edge 36 at the tip of the head 30.

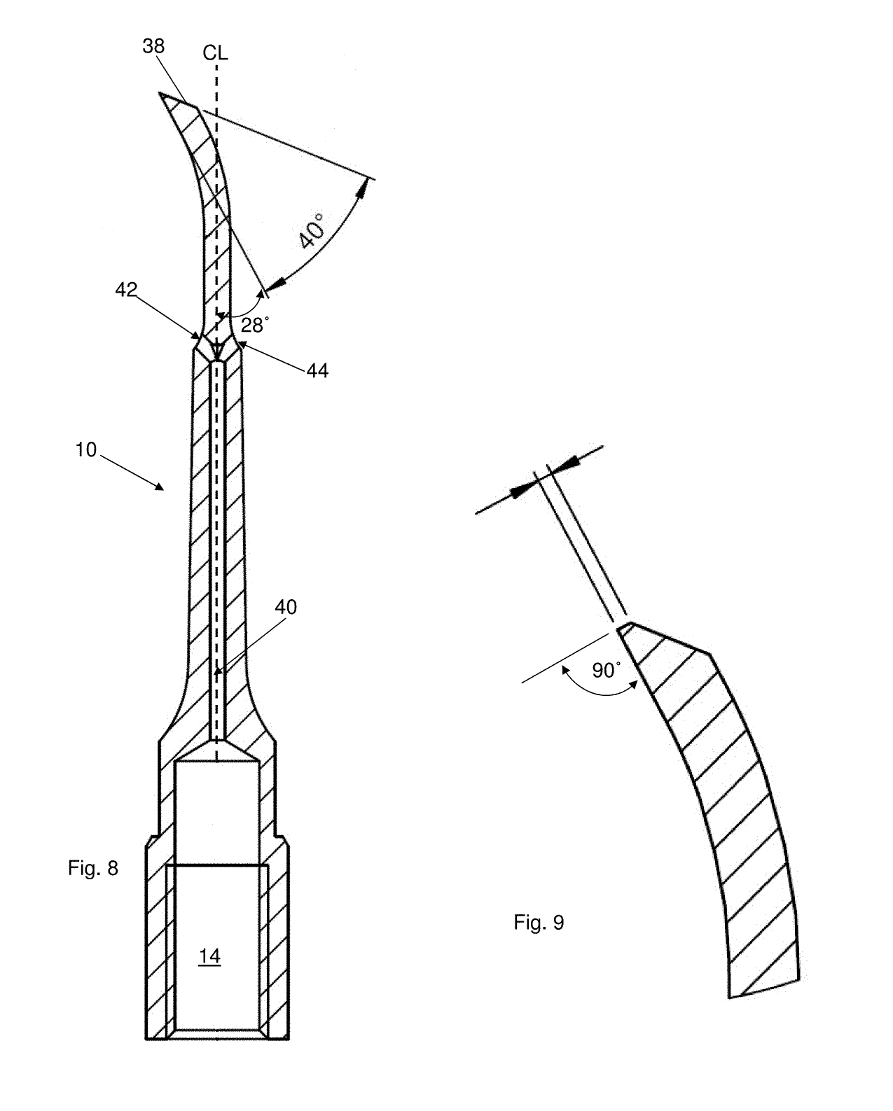

As is shown in FIG. 8, the angle between the front face of the head 30 and the centerline, which is coaxial with the longitudinal axis of the shaft 16, is preferably about 28 degrees, but may vary between 26-30 degrees, or between 23-33 degrees. An angled surface 38 is formed on the rear of the head 30, adjacent the edge 36, and strikes an angle of about 40 degrees with the front face of the head 30, as shown in FIG. 8. The angle between the front face of the head and the angled surface 38 is preferably about 40 degrees, and may vary between 38-42 degrees, or between 35-45 degrees. The rear face of the head 30 is preferably curved to match the curvature of the front face of the head 30, and thus the angled surface 38 creates a tapering down in the thickness of the head 30 at the end of the head 30 farthest from the proximal end 12. This creates a “wedge effect” that allows the insert 10 to be slipped into narrow passages during use.

A curved portion 34 is formed between the shaft end 32 and the edge 36 on the front face of the head 30, and between the shaft end 32 and the angled surface 38 on the rear face of the head 30. The curved portion 34 has rounded lateral sides 34a and 34b, and a radius of curvature along the longitudinal axis of the insert 10 of about 6.0 millimeters, but this can vary by at least about 1-3 millimeters greater or lesser. The corresponding curvatures at the front and rear faces of the head 30 maintain a consistent thickness of the portion of the head 30 that extends from the shaft end 32 to the angled surface 38, which is then followed by a tapered, decreasing thickness to the edge 36. This consistent thickness of the head 30, which may be about 1.0 millimeter+/−0.1 millimeters, allows insertion of the head 30 past the tapered surface 38 into narrow spaces during use.

It should be noted that the sizes of the portions of the insert 10 described above are defined in functional terms based on how the insert responds to ultrasonic vibrations and surgical action (back-and-forth movement, etc.). For example, the preferred insert 10 may be made of stainless steel, titanium or an alloy with a titanium nitride coating, and this material is cryogenically heat-treated by quenching and stress-relieving to obtain a hardness of between 57-61 HRC. Any alternative material and treatment that is suitable may be used instead. The response to ultrasonic vibrations of an insert made of this material and useful for human surgeries generally includes resonating at a frequency between about 25 kHz and about 29 kHz and a longitudinal direction of the movement that is triggered by the vibrations. Response to surgical action also includes the ability of the insert 10 to operate efficiently, according to its overall size and the sharpness of its operative portion, such as the edge 36. In this regard, a critical length in relation to the vibrating system of the transducer and the insert may be 32.6 mm+/−1 mm and a functional angle of the operating portion of about 28°+/−5°, as described above. In the embodiment of FIG. 1, the efficiency of the insert derives from the acuteness of the angle at the edge 36 and the front face of the head 30 (preferably at 90°+/−5°, as described above) and the width of the edge 36 (preferably 0.2 mm). It should be noted that the “+/−” symbols indicate the range within which functionality requirements are still met. If an insert that is designed to be used on larger or smaller patients, it will become apparent to the person of ordinary skill how to adapt the sizes described herein in order to adapt the invention's sizes and angles for those purposes.

At the shaft end 32 of the head 30 there are apertures 42 and 44 in fluid communication with an irrigation channel 40 that extends at least a portion of the length of the insert 10 from the bore 14, along the shaft 16 and into the head 30. Conventional piezoelectric surgical tools are equipped to force water into an insert, and this channel 40 receives pressurized water from the tool. The channel 40 splits adjacent the shaft end 32 of the head 30 and opens to the apertures 42 and 44 to allow irrigation fluid that is pumped into the bore 14 to flow out of the apertures 42 and 44. The irrigation system, made up of the channel 40 and apertures 42 and 44, causes irrigation fluid, such as water, to flow to both sides of the head 30 during use. Because the apertures 42 and 44 are mounted on opposite sides of the head adjacent the shaft end 32, liquid, such as water, may be sprayed or otherwise conveyed out of the apertures 42 and 44 to the surgical site, thereby cooling the tissue and the head 30. The apertures 42 and 44 may be on the shaft or on the front and rear faces of the head 30 as shown. The apertures may not be opposing, but this is preferred.

As shown best in FIG. 3, the head 30 is laterally narrower near the shaft end 32 than adjacent the edge 36. Furthermore, the curvature of the body 30 through the thickness direction causes the apex of the edge 36, which is formed at the tip of the head 30, to be located at the extreme end of the insert 10 which is also the point of the head 30 farthest from a centerline of the shaft. This configuration allows a surgeon to position the edge 36 along and around a curved outer surface, such as a cylinder, a tooth, a bone or other structure.

The use of the insert 10 will now be described in association with FIG. 11 and the other illustrations. The insert 10 is attached to the piezoelectric surgical device (not shown), and the device is activated to vibrate the tip at high frequency, preferably between about 24 and 36 KHz, and simultaneously at very low frequency, such as below 60 Hz. The edge 36 is placed against an incision formed in tissue 100 that extends entirely to the bone. In a preferred embodiment, the front face of the head 30 faces toward the bone, as shown in FIG. 11, so that the curvature of the curved portion 34 allows the edge 36 to extend around the curved bone, tooth or other structure, if needed to place the edge 36 against the incision. Alternatively the insert 10 can be rotated from this position about the longitudinal axis (which is coaxial with the centerline CL in FIG. 8), in a range from a few degrees up to 90 degrees. In a preferred embodiment, the angle between the longitudinal axis of the insert 10 and the path that the edge 36 follows against the bone may be in a range from a few degrees to about 90 degrees, more preferably from about 10 degrees to about 45 degrees, and most preferably around 15 to 30 degrees. The path may be the incision formed through the periosteum, or it may be the longitudinal axis of the bone, or another path. The amount of force applied along the longitudinal axis may be between 2.0 and 6.0 Nm.

The edge 36 extends into the incision and, due to the high frequency vibrations of the insert 10, the edge 36 further opens the existing opening between the bone and the periosteum. With some back-and-forth movement of the edge 36 along the opening in the periosteum, the periosteum is separated from the bone due to the edge 36 being driven by high-frequency, low-amplitude movements in the lateral and longitudinal directions. The edge 36 does not cut the periosteum, but rather is inserted between the periosteum and the bone, and continues to separate the periosteum from the bone along the path through which the edge 36 is conveyed by the surgeon. These back-and-forth movements, along with forces applied by hand in the axial direction (along the axis of the shaft 16) and/or the lateral direction, cause the edge 36 to further insert between the bone and the periosteum by separating the periosteum as a layer from the bone. Further movement along the incision causes further insertion of the head 30, so that with a gentle but firm back-and-forth movement, while maintaining the edge 36 in contact with both the periosteum and the bone at the junction of the two, the vibratory force of the edge 36 at the point where periosteum attaches to bone extends the separation. No tissue layers are incised by the insert 10, but the periosteum remains intact and separates as an intact layer from the bone.

As noted above, the openings 42 and 44 permit irrigation fluid, such as water, to flow, spray (such as by atomization) or otherwise contact the tissue at and near the front face and the rear face of the head 30 at and near the edge 36. The water preferably contacts both the bone and the periosteum to reduce their temperature, lubricate the edge 36, avoid dehydration of the tissue, and flush loose fragments of tissue away from the site where the separating of periosteum from bone occurs.

It should be pointed out that the edge 36 is always maintained to remain sharp enough to separate the periosteum from the bone, but not sharp enough to incise through the periosteal layer without misuse of the insert 10 by the surgeon. To have these characteristics in the insert 10 described herein, the edge 36 is always about 1.0 millimeter wide or wider, and is not sharpened sufficiently to incise the periosteum.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. An improved removable insert for use with a vibrating surgical tool, the insert having a proximal end for mounting to the vibrating surgical tool and a shaft extending from the proximal end, the improvement comprising a head mounted to the shaft, the head having a head thickness substantially less than a head width and a curvature through the head thickness, the terminal end of the head defining an edge for disposing against an incision between bone and a periosteum covering the bone.

2. The improved removable insert in accordance with claim 1, further comprising at least one liquid passage extending through the shaft and a liquid-dispensing aperture formed on at least one side of the insert, and in fluid communication with said at least one liquid passage, for dispensing liquid from the liquid-dispensing aperture.

3. The improved removable insert in accordance with claim 2, further comprising a second liquid-dispensing aperture in fluid communication with said at least one liquid passage on a second side of the insert opposite the first side for dispensing liquid from the second liquid-dispensing aperture.

4. The improved removable insert in accordance with claim 1, further comprising at least one liquid passage extending through the shaft and a liquid-dispensing aperture formed on the head adjacent the shaft, and in fluid communication with said at least one liquid passage, for dispensing liquid from the liquid-dispensing aperture.

5. The improved removable insert in accordance with claim 4, wherein the liquid-dispensing aperture is on a front face of the head, and a second liquid-dispensing aperture is in fluid communication with said at least one liquid passage on a rear face of the head for dispensing liquid from both of said apertures.

6. The improved removable insert in accordance with claim 1, wherein the edge is formed on a surface that is curved from one lateral side to an opposite lateral side.

7. The improved removable insert in accordance with claim 1, wherein the edge extends from a front face to a tapered rear surface.

8. The improved removable insert in accordance with claim 7, wherein the edge is at least about 1.0 millimeter wide.

9. The improved removable insert in accordance with claim 1, wherein a front face of the head forms an angle with a longitudinal axis of the shaft of about 28 degrees.

10. A method of separating at least a portion of a periosteum from a bone, the method comprising:
(a) mounting a removable insert on a vibrating surgical tool, the insert having:
(i) a proximal end mounted to the vibrating surgical tool;
(ii) a shaft extending from the proximal end, and
(iii) a head mounted to the shaft, the head having a head thickness substantially less than a head width and a curvature through the head thickness, the terminal end of the head defining an edge;
(b) vibrating the tip;
(c) disposing the edge against an incision between bone and a periosteum covering the bone; and
(d) manually displacing the insert relative to the incision, thereby moving the edge along the incision.

11. The method in accordance with claim 10, further comprising forcing a liquid through the shaft and out of the insert adjacent the head.

12. The method in accordance with claim 10, further comprising disposing a longitudinal axis of the shaft at an angle of between about 0 and 90 degrees relative to the incision.

13. The method in accordance with claim 12, further comprising disposing the longitudinal axis at an angle of between about 15 and 45 degrees relative to the incision.

14. The method in accordance with claim 12, further comprising forcing a liquid through the shaft and out of the insert adjacent the head.

15. The method in accordance with claim 12, further comprising forcing a liquid through the shaft and out of the insert on opposing sides adjacent the head.
